(19) United States
(12) Patent Application Publication
(10) Pub. No.: US 2026/0142493 A1
(43) Pub. Date: May 21, 2026

(54) UNINTERRUPTIBLE POWER SUPPLY SYSTEM

(71) Applicant: Zopec Medical LLC, Blaine, MN (US)

(72) Inventor: Strong Shihchuang HUANG, Blaine, MN (US)

(73) Assignee: Zopec Medical LLC, Blaine, MN (US)

(21) Appl. No.: 19/394,579

(22) Filed: Nov. 19, 2025

Related U.S. Application Data
(60) Provisional application No. 63/722,390, filed on Nov. 19, 2024.

(51) Int. Cl. H02J 9/06; A61F 7/00; A61G 11/00; H02J 7/00; H02J 7/02; H02J 7/80

(52) U.S. Cl. CPC H02J 9/062; A61F 7/007; A61G 11/00; H02J 7/02; H02J 7/80; H02J 7/865

(57) ABSTRACT

An uninterruptible power supply system having a housing, at least one input port configured to receive power from an external power source, at least one output port configured to provide power to a medical device, and a battery pack contained within the housing. The power supply also having a power supply circuit electrically coupled to the input port, output port, and battery pack. The power supply also having a controller that controls the power supply circuit and is configured to select between a pass-through mode, a back-up power mode, and an inverter mode.

### CROSS-REFERENCE TO RELATED APPLICATIONS

This application claims the benefit of U.S. Provisional Ser. No. 63/722,390, filed on Nov. 19, 2024, the disclosure of which is hereby incorporated by reference in its entirety. To the extent appropriate a claim of priority is made to the above-listed application.

### BACKGROUND

Hospitals utilize numerous medical devices which require constant power. For instance, conventional infant warmer devices may be used to provide a heated environment for infants. Infants born prematurely may have difficulty sustaining a proper body temperature. Often these medical devices are plugged into an external power source such as wall outlets or other power receptacles. However, power receptacles may cease functioning. Further, patients connected to these medical devices may need to be transported. The medical devices may be unable to be moved due to concerns with power supply to the medical device. If these devices cease operating, critical care being provided to patients could be interrupted or stopped. In the case of an infant warmer, lack of power will prevent the infant warmer from performing its intended functions of regulating the environment around an infant, for example.

### SUMMARY

In general terms, this disclosure is directed to an uninterruptible power supply system. In some embodiments, and by non-limiting example, the uninterruptible power supply system is part of an uninterruptible medical system, as described herein.

An aspect of the present disclosure relates to an uninterruptible power supply comprising: a housing; at least one input port configured to receive power from an external power source; at least one output port configured to provide power to a medical device; a battery pack contained within the housing; a power supply circuit electrically coupled to the input port, output port, and battery pack; and a controller that controls the power supply circuit and is configured to select between a pass-through mode, a back-up power mode, and an inverter mode.

Another aspect of the present disclosure relates to a method of transporting an uninterruptible power supply, the method comprising: in a first location, selecting an inverter mode of an uninterruptible power supply; disconnecting the uninterruptible power supply from an external power source; transporting the uninterruptible power supply to a second location; connecting the uninterruptible power supply to a second external power source or power receptacle.

Another aspect of the present disclosure relates to a method of operating an uninterruptible power supply, the method comprising: selecting an inverter mode of the uninterruptible power supply; disconnecting a first switch delivering power from an external power source to a pass-through path and a second switch receiving power from the pass-through path, the second switch providing power to an electronic device; connecting a third switch delivering power from the external power source to a battery pack path and connecting the second switch to the battery pack path to provide power the electronic device.

### DETAILED DESCRIPTION OF THE DRAWINGS

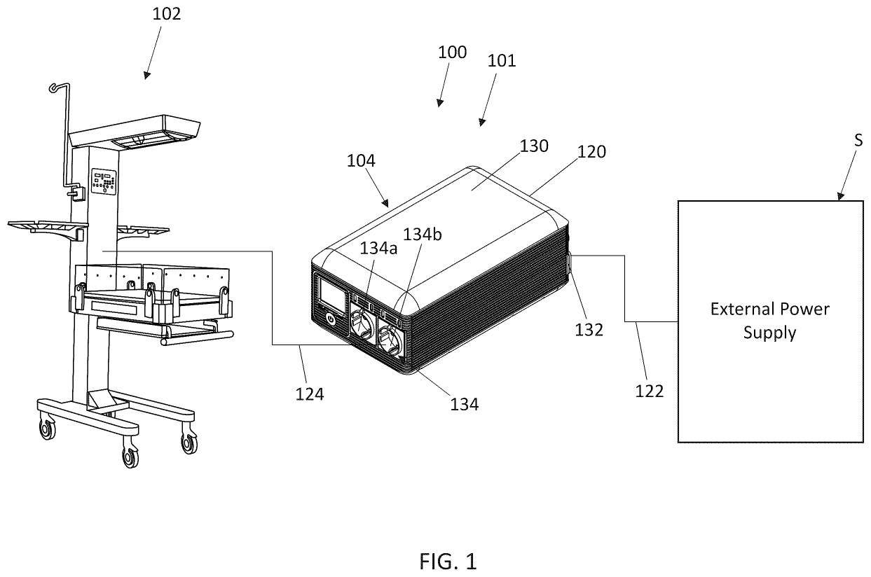

FIG. 1 is a perspective view of an example uninterruptible medical system.

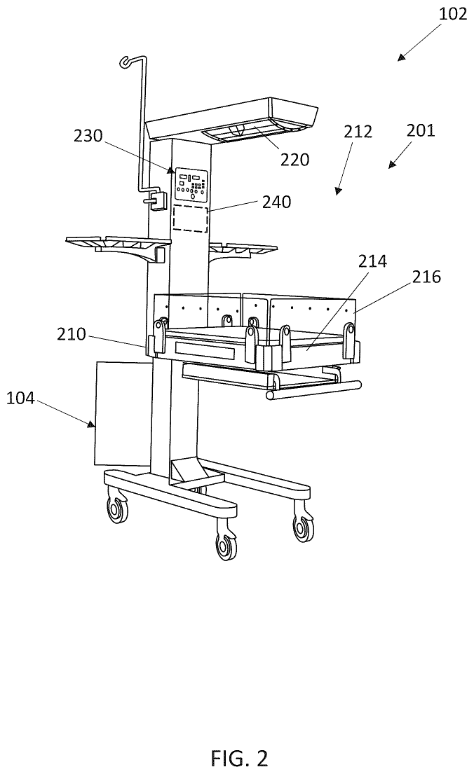

FIG. 2 is a diagram illustrating an example infant warmer device.

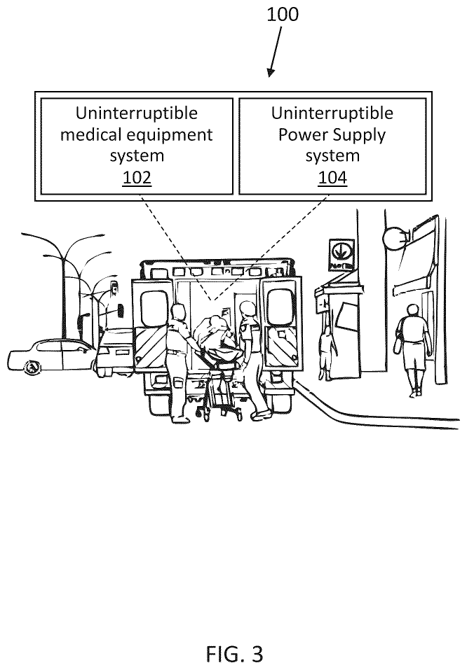

FIG. 3 is a perspective view of an example patient transport using the power supply system.

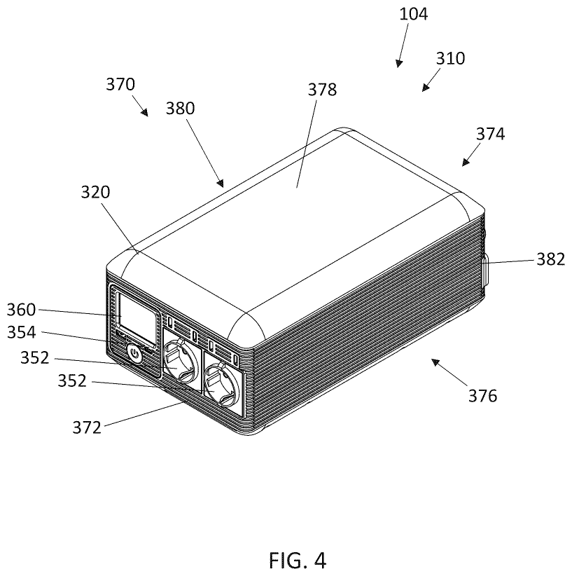

FIG. 4 is a perspective view of an uninterruptible power supply system.

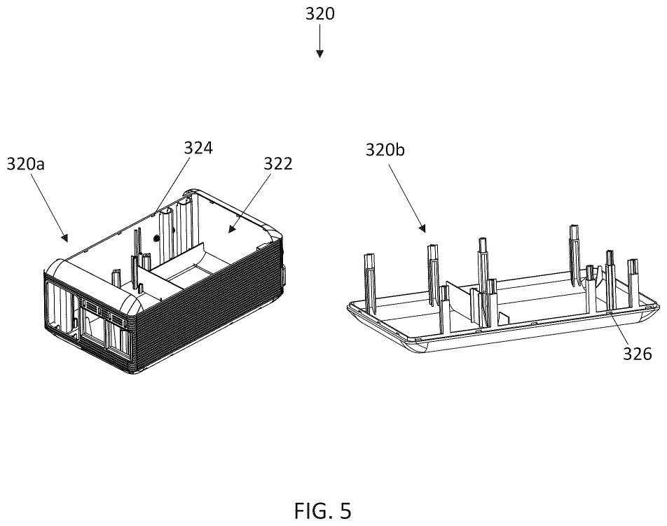

FIG. 5 is a perspective view of two halves of a housing of the uninterruptible power supply system of FIG. 4.

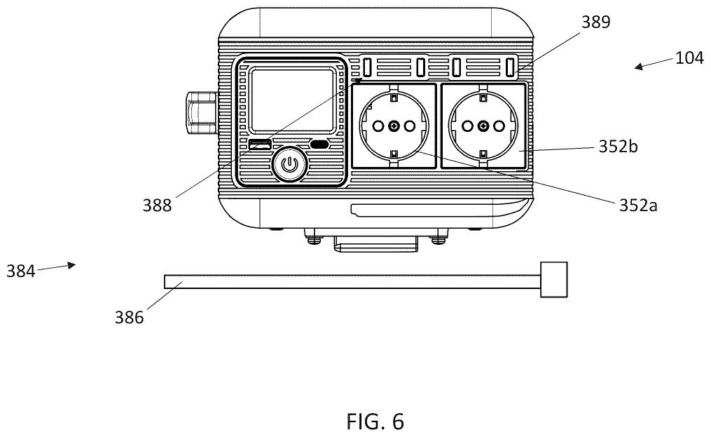

FIG. 6 is a side view of a front side of the uninterruptible power supply system of FIG. 6.

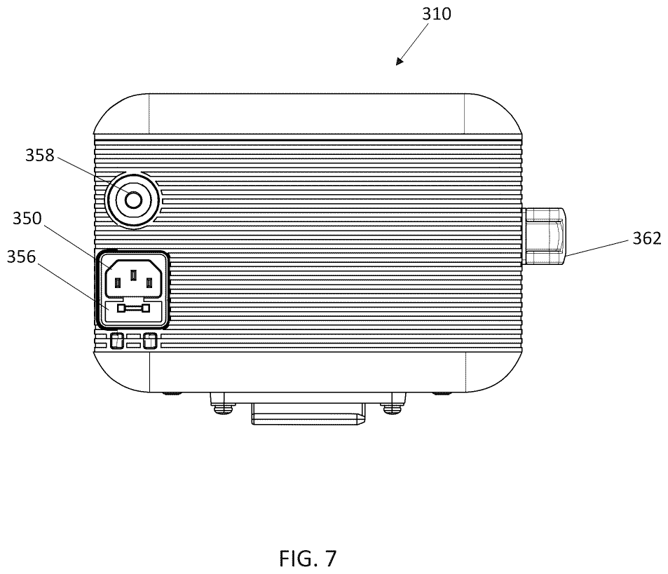

FIG. 7 is a side view of a fuse panel of the uninterruptible power supply system of FIG. 5.

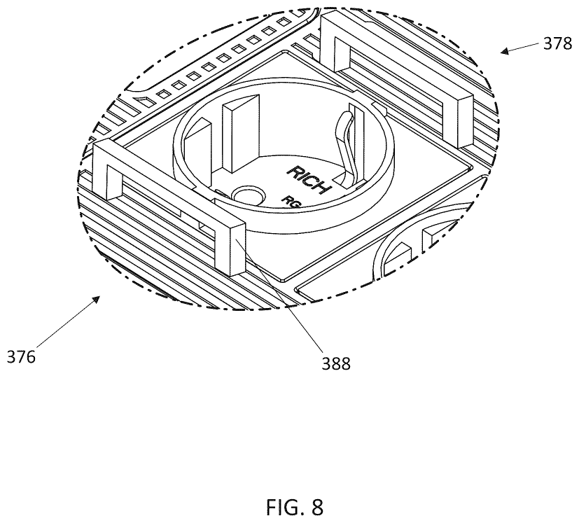

FIG. 8 is a perspective view of a locking mechanism on the front side of the uninterruptible power supply system of FIG. 5.

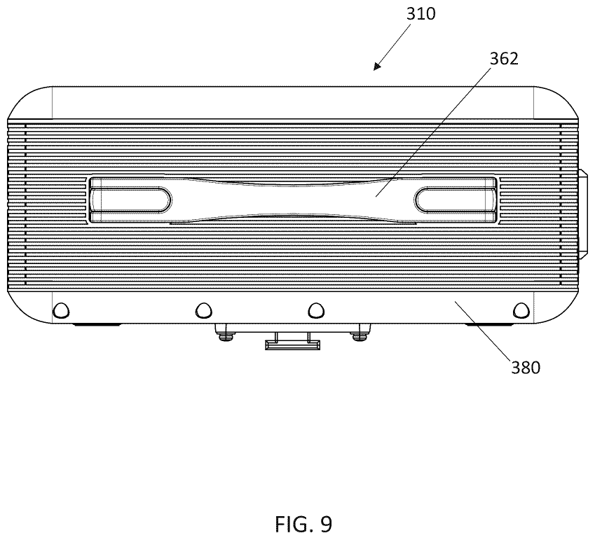

FIG. 9 is a side view of a handle on the left side of the uninterruptible power supply system of FIG. 5.

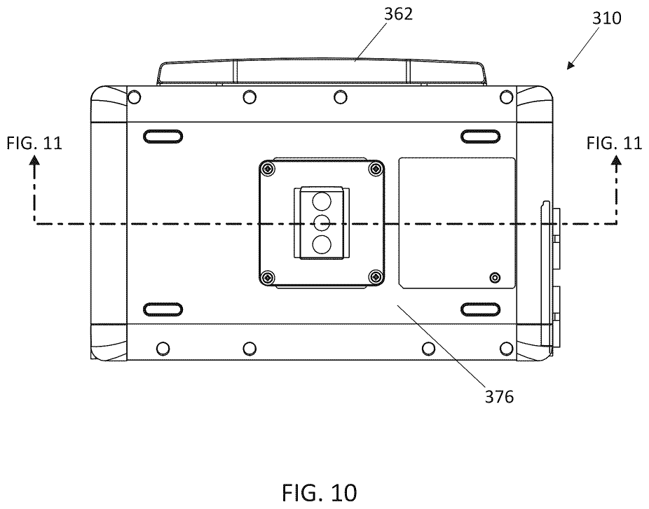

FIG. 10 is a side view of a bottom side of the uninterruptible power supply system of FIG. 6.

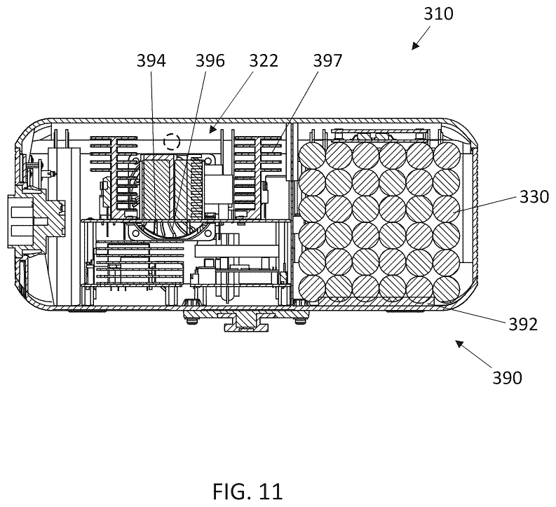

FIG. 11 is a cross sectional view of an example cooling system of the uninterruptible power supply system of FIG. 5.

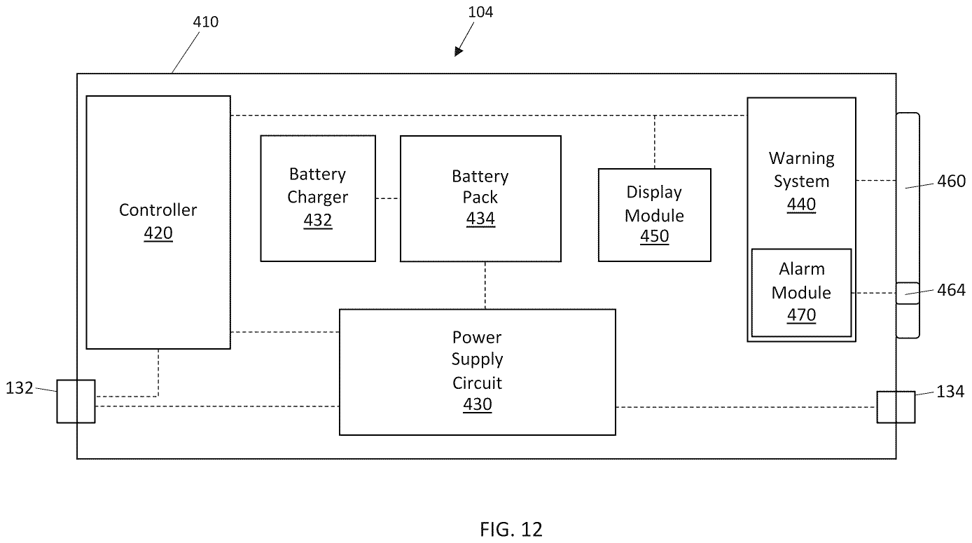

FIG. 12 is a diagram illustrating an example uninterruptible power supply.

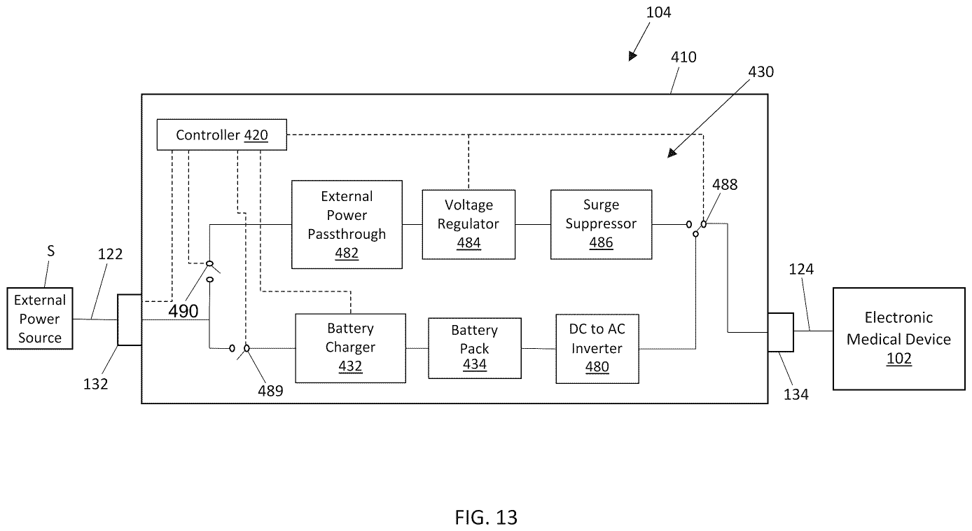

FIG. 13 is a schematic of an example power supply circuit.

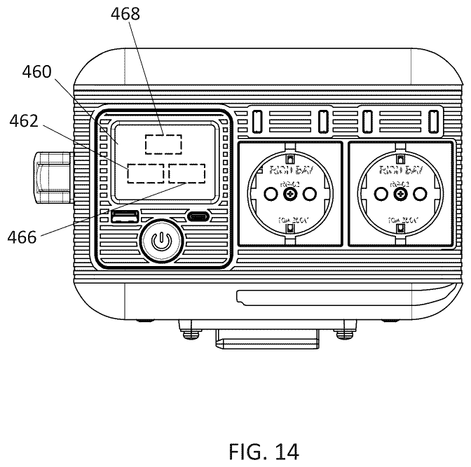

FIG. 14 is a front perspective view of the display of the uninterruptible power supply system of FIG. 5.

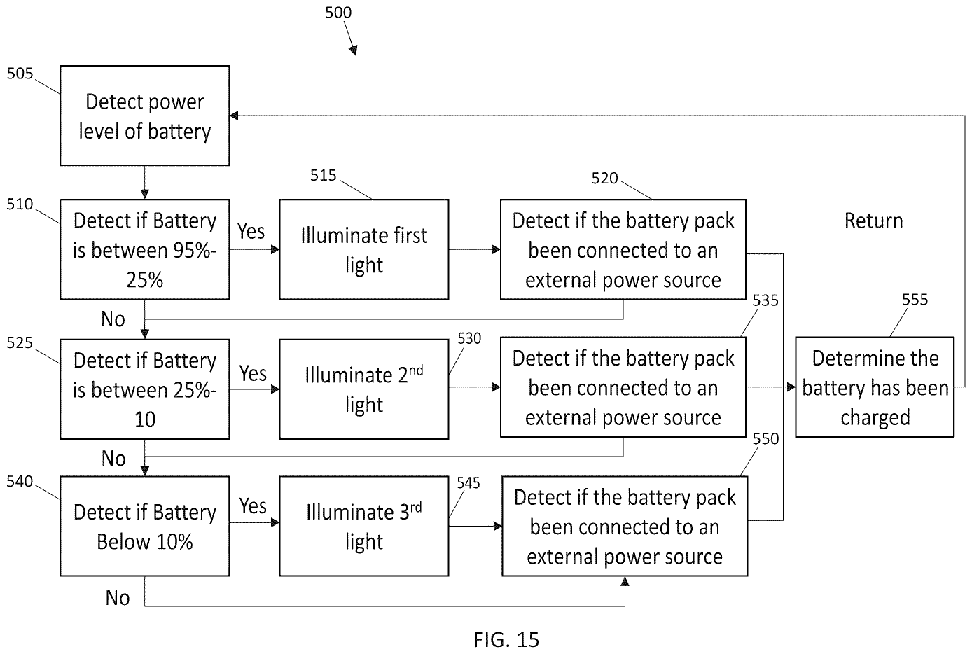

FIG. 15 illustrates an example method of controlling the illumination of the display.

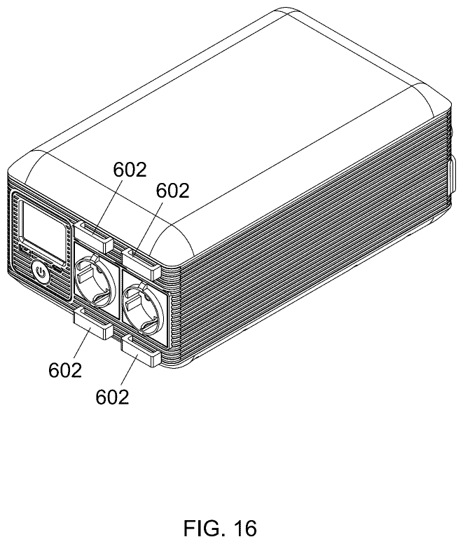

FIG. 16 illustrates an alternate locking mechanism for an uninterruptible power supply system.

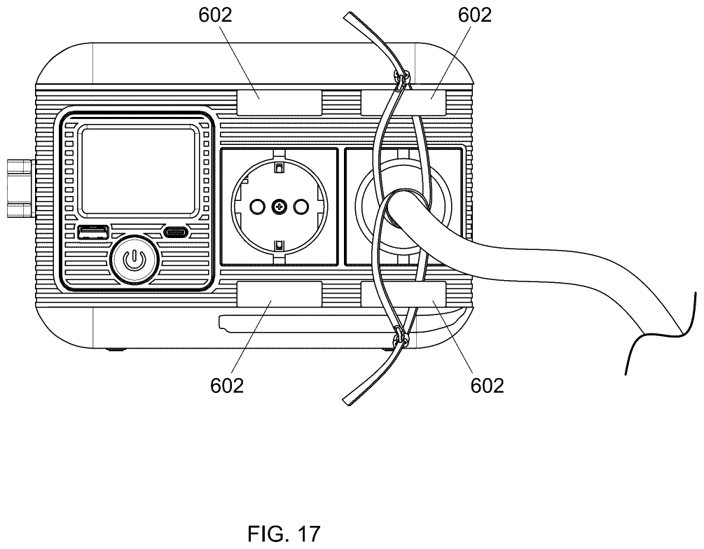

FIG. 17 illustrates the locking mechanism for the uninterruptible power supply system of FIG. 16.

### DETAILED DESCRIPTION

Various embodiments will be described in detail with reference to the drawings, wherein like reference numerals represent like parts and assemblies throughout the several views. Reference to various embodiments does not limit the scope of the claims attached hereto. Additionally, any examples set forth in this specification are not intended to be limiting and merely set forth some of the many possible embodiments for the appended claims.

FIG. 1 is a perspective view of an example uninterruptible medical system 100. An example uninterruptible medical system 100 can be an uninterruptible medical equipment system 101 for use in maintaining power supply to medical equipment. The example uninterruptible medical equipment system 101 includes an electronic medical device 102 and an uninterruptible power supply system 104. The example uninterruptible power supply system 104 includes a power supply 120, a power cable 122, and an output cable 124. The example power supply 120 includes a housing 130, at least one input port 132 (shown in FIG. 6), and at least one output port 134. An external power source S is also shown in FIG. 1.

In the illustrated example, the example uninterruptible medical equipment system 101 includes an electronic medical device 102 and an uninterruptible power supply system 104. The example electronic medical device 102 may be an infant warmer which provides a heated environment. The electronic medical device 102 can be various other medical devices as well, and the infant warmer device is one such example of an electronic medical device. If power is lost while the electronic medical device 102 is in use, the electronic medical device 102 cannot operate to assist the infant in regulating body temperature. In order to avoid such disruptions, the uninterruptible medical system 100 includes the uninterruptible power supply system 104 that functions to maintain a continuous supply of power to the electronic medical device 102 while the device is in use (such as during a power outage or during patient transport), so that the operation of the electronic medical device 102 is not interrupted.

The electronic medical device 102 operates to provide a controlled and safe environment for newborn infants, particularly those requiring additional warmth and care. In some embodiments, the electronic medical device 102 generates heat to provide an environment which is close to the condition an infant would experience in the womb, promoting growth, development, and recovery. For example, the infant warmer may be necessary for premature infants, infants with low birth weights, or difficulty regulating body temperature. Examples of the electronic medical device 102 are illustrated and described in further detail herein with reference to FIGS. 2-4.

The uninterruptible power supply system 104 provides uninterrupted power to the electronic medical device 102 to maintain the continuous operation of the electronic medical device 102 even in the absence of an external power source S, or in the event that power is lost from the external power source S. The example uninterruptible power supply system 104 includes a power supply 120, a power cable 122, and an output cable 124. The power supply 120 includes at least one input port 132 (shown in FIG. 7) and at least one output port 134 (shown in FIG. 5).

The power supply 120 is the portion of the uninterruptible power supply system 104 that receives, stores, and outputs power to the electronic medical device 102. The example power supply 120 includes a battery pack, which can be charged with power from the external power source S and can subsequently supply the stored power to the electronic medical device 102. In some embodiments, when the power supply 120 is connected to the external power source S, the power supply 120 supplies power to the electronic medical device 102, while bypassing the battery pack. In some embodiments, the power supply 120 delivers power from an external power source S and from the battery pack, such as to collectively provide even greater power output, whereas in other embodiments the power supply 120 delivers power selectively from either the external power source S or from the battery pack. The power supply 120 is configured to have a peak supply of at least 1200W and a continuous power supply 750W. The power supply 120 is configured to operate for 500-900 Watt hours. In other examples, the power supply is configured to operate for 600-800 Watt hours. In other examples, the power supply is configured to operate for 700 Watt hours. In other examples, the power supply 120 may be configured to have a peak supply of at least 2400W and a continuous power supply of 1000W.

The input port 132 provides a connection for receiving the power cable 122, to receive power from the external power source S. The output port 134 provides a connection for receiving an output cable 124 of the electronic medical device 102, to supply power from the power supply 120 to the electronic medical device 102. The power supply 120 is illustrated and described in further detail with reference to FIGS. 5-16.

The power cable 122 receives power from an external power source S for delivery to the power supply 120. The AC power is then supplied to the power supply 120 directly with the power cable 122. In some examples, the external power source S can be one or more additional uninterruptible power supply system 104 which are daisy chained to enlarge the available level of stored energy. Advantageously the total available level of energy is increased and provides a larger length of time for patient transport. The daisy chaining will be described in greater detail later.

The present disclosure refers to example embodiments in which the uninterruptible power supply system 104 provides power to the electronic medical device 102. In other embodiments, the uninterruptible power supply system 104 can instead provide power to other electronic devices other than the electronic medical device 102. The electronic device can include a device other than a medical device, such as a non-medical device. Accordingly, within the present disclosure, the term medical system 100 can, when the context permits, be replaced with the terms electronic system or medical device system to describe other embodiments that are within the scope of the present disclosure. Additionally, the term infant warmer device 102, when the context permits, can be replaced with the terms electronic device or medical device to form additional embodiments according to the present disclosure. In the present disclosure, it should be understood that different examples may include the same components as other examples which may not be redescribed.

FIG. 2 is diagram illustrating an electronic medical device 102 connectable to the uninterruptible medical system 100. In this example, the electronic medical device 102 may be an infant warming device 201. The infant warming device 201 includes a main body 210, a heating element 220, a display 230, and a controller 240. The main body 210 includes a power cable port (not shown), a warming region 212, and the display 230.

The main body 210 is configured to provide a structure for holding an infant (such as a bed, mattress, or bassinet), and components needed to heat an environment. The heating element 220 is provided to heat the environment holding the infant. The display 230 is used to update medical staff of the operating conditions of the infant warmer and the vital signs of the infant. The display 230 may also include warning systems to alert medical staff if the infant warming device 201 is operating outside of set conditions or the infants monitored vital signs are outside expected parameters. The controller 240 is configured to control the display and heating element 220.

The example electronic medical device 102 is powered by the output cable 124 inserted into power cable output port 134 of the uninterruptible power supply system 104. The power is used to power the electronic medical device 102. In the example electronic medical device 102, the controller 240, the display 230 and the heating element 220 may be powered by the uninterruptible power supply system 104 or the exterior power source S. The electronic medical device 102 requires continuous power supply due the expended energy necessary to continuously run the heating element and other components of the infant warming device 201. As such, the uninterruptible power supply system 104 may be used to allow power from the external power source to be passed-through the power supply 120 or provide stored power within the power supply 120 directly to infant warming device 201 or another electronic medical device 102.

The main body 210 is a structure for holding the heating element 220, display 230, and controller 240. As such, the main body 210 defines the warming region 212. The warming region 212 retains the heating element 220. In the example infant warming device 201, the heating element 220 may be a heater. The warming region 212 further includes a surface 214 configured to hold an infant. The main body 210 may further define sidewalls 216 extending upward from the surface 214 which prevent the baby from falling off the surface. The sidewalls 216 further assist in retaining heat. The sidewalls 216 also assist in maintaining a sterile environment.

Referring to FIG. 2, in the example electronic medical device 102, the electronic medical device 102 includes an attachment region. The attachment region is configured to receive the power supply system. In some instances, the attachment region may be a bracket (shown in FIG. 10). The power supply system 104 may be mounted on the medical device by a bracket. In other instances, the attachment region may be a surface where the power supply system rests. Alternatively, the uninterruptible power supply system 104 may be mounted on a wall or another surface adjacent the electronic medical device 102.

FIG. 3 is a perspective view of an uninterruptible medical system 100 used in the transport to an emergency transport vehicle. For instance, as shown in FIG. 3, patients may be in a critical condition within an intensive care unit and already utilizing a medical device connected to the uninterruptible power supply system 104. The patient may need to be transported from one hospital to another or locally within the hospital, requiring the medical device to be disconnected from an external power source of the hospital. The power supply system 104 may provide power to the electronic medical device 102 after disconnection from the external power source and allow the device to operate without interruption. The patient is transported to an emergency transport vehicle where the power supply system 104 can reconnect to a power source of the emergency transport vehicle. Further, the power supply system may be reconnected to an external power source, be recharged, and the medical device can resume being power by a new external power source without interruption. The example emergency transport vehicle is an ambulance; however, the vehicle can be any type of vehicle, such as a helicopter. In other examples, the medical system 100 could be used on its own either in a hospital or a home. In other examples, the medical system 100 could be used within the ambulance or transportation out of an ambulance.

The example power supply system 104 receives signals as a pure sine wave, which can be accepted from emergency transport vehicles without damaging the vehicle's power source. A square waveform supplied to the ambulance by conventional medical grade power supply devices could damage the ambulance, and a pure sine wave can avoid such disadvantages. Additionally, the uninterruptible power supply system 104 has a weight of no greater than 17 lbs. Conventional medical grade power supply devices weigh as much as 50 lbs. or more. In some examples, the uninterruptible power supply system 104 has a weight within 4-17 lbs. In some examples, the uninterruptible power supply system 104 has a weight within 12-16 lbs. Advantageously, the uninterruptible power supply system 104 allows a lightweight system capable of being easily transported in emergencies or around the medical facilities. For instance, medical staff may desire to the move the electronic medical device 102 to another location within a care facility such as a different area or room within a hospital. The uninterruptible medical system 100 allows movement of the electronic medical device 102 to other locations without having to disconnect power to the infant warming device 201. It is understood that other scenarios of patient transport can arise.

FIG. 4 illustrates an example perspective view of the uninterruptible power supply system 104. FIG. 5 is a perspective view of two halves of a housing 320 of the uninterruptible power supply system 104 of FIG. 4. FIGS. 6-11 illustrates various example side views and cross sectional views of the uninterruptible power supply system 104. The power supply 310 may be switched between operational modes based on the whether external power is available. The power supply 310 includes a housing 320, a battery pack 330 and a controller (not shown). The housing 320 retains the battery pack 330. The housing 320 is flame retardant and explosion proof. The housing 320 can include an input port 350, two AC output ports 352a, 352b, USB ports 354, a fuse panel 356, a reset button 358, a display 360, a handle 362, a plurality of sides 370, a locking mechanism 384, and a cooling system 390 (shown in FIG. 13). Referring to FIG. 5, the plurality of sides 370 of the housing 130 may include a front side 372, a back side 374, a bottom side 376, a top side 378, a left side 380, and a right side 382. The plurality of sides 370 define the exterior of the housing 320 and facilitate the positioning of the housing 130 and cables 122, 124 for the power supply 310. The front side 372 may be opposite the back side 374, the bottom side 376 may be opposite the top side 378, the left side 380 may be opposite the right side 382.

The battery pack 330 (shown in FIG. 11) is configured to store power for powering the electronic medical device 102. The battery pack 330 can be a plurality of lithium ion battery or a plurality lithium ion battery packs. Lithium ion battery packs reduce the weight of the battery pack which contributes to the reduced weight of the power supply system 104. As previously discussed, conventional medical grade power supply devices weigh as much as 50 lbs. or more.

The housing 320 can be at least two pieces 320a, 320b (shown in FIG. 5) joined together to form an interior 322. The piece 320a may be the main body of the housing. The piece 320b may be a cover joinable to the top of the piece 320a. The two pieces 320a, 320b may be joined together as a clamshell housing. In the example, housing 320, the two pieces 320a, 320b may include tabs 324 and retaining regions 326 which receive the tabs on the interior 322 of the housing. The tabs 324 allow the two housing to engage and remain shut; however, in some instances the tabs 324 can become damaged when the housing 320 is disassembled. The two pieces 320a, 320b are further joined together by fasteners. The fasteners ensure the two pieces are properly joined.

The USB ports 354 (shown in FIG. 6) are configured to output power through USB-C cables. The USB ports 354 allows the uninterruptible power supply system 104 to pair with different secondary devices which are connected to the power supply 310 separately from the electronic medical device 102. An example secondary device can be a mobile device or another medical device. The USB-C port may form a communication link between the secondary device and the power supply system 104 and then power the secondary device. The link allows the secondary device and power supply 310 to determine a required voltage for the secondary device or devices and supply the required voltage to the secondary device. The USB-C can detect whether the required voltage is at least one of 5 volts, 9 volts, 20 volts, 24 volts, or 30 volts to be supplied to the secondary device.

The fuse panel 356 (shown in FIG. 7) is configured to movably allow or restrict access to a fuse of the power supply. The fuse panel 356 is configured to allows a user to remove the panel for the replacement of the fuse without opening the housing 320. The fuse panel 356 is positioned on the back side 374 of the housing 320. In particular, the fuse panel 356 may be positioned above the input port 350 on the back side 374.

The reset button 358 (shown in FIG. 7) is configured to reset the power supply 310. Resetting may be used after a power outage if switching the power supply between operational modes has become prevented. As will be explained in greater detail later, in some instances, the power supply may remain in a back-up mode. The back-up mode allows the power supply 310 to act as the power source for the uninterruptible medical equipment system 101. As such, the reset button 358 may allow a user to reset the power supply and the controller to return to a pass-through mode where the external power source S power the uninterruptible medical equipment system 101.

The example display 360 provides an indication of the power level of the battery. The display 360 is a touch screen display. In the example, the touch screen is illuminated by an LED lights. The entire touch screen may illuminate different colors based on the remaining power of the power supply. The different colors may indicate different levels of power remaining to a user. Further, the brightness of the LED lights may change with different levels of power remaining. For instance, below a threshold power remaining, the LED lights may be adjusted to be lower. The handle 362 (shown in FIG. 9) is configured to allow the uninterruptible power supply system 104 to be held and moved. As the power supply 310 is a medical grade power supply weighing approximately 15 lbs, the handle allows a user to easily hold the weight of the power supply 310. Medical grade means compliant with standards IEC-60601-1, and 62133-2. As such, the power supply 310 does not emit electromagnetic fields which substantially interfere with other medical devices and the power supply 310 is made of a fire retardant material making the power supply 310 fireproof. The housing of the power supply 310 meets UL94 V0 ratings. For instance, the fire retardant material meets a test of requiring a vertical portion of the material to stop burning within 10 seconds and does not drip particles.

The locking mechanism 384 (shown in FIG. 6) is configured to lock the cables to the power supply. The locking mechanism 384 allows the locking of power cables to the housing 320 and prevents undesired removal of power cables from the power supply. Advantageously, power supply to cables inserted into the input and output ports of the power supply are prevented from being disrupted.

The cooling system 390 (shown in FIG. 11) provides a mechanism for managing heat generated by the battery pack and other electrical components within the power supply 310. The battery pack generates heat during operation and must be cooled. The cooling system 390 is configured to prevent overheating of the battery pack.

In some examples, the uninterruptible power supply system further includes a silicon cover around the housing. The cover may provide water-resistance and protect the housing if dropped.

FIGS. 6 and 7 illustrate example front and back sides of the power supply system 104. The front side 372 and back sides 374 may include various input and output ports. In the example front side 372 of FIG. 6, the housing 320 includes an input port 350. In the example back side 374 of FIG. 7, the back side includes two AC output ports 352a, 352b, the USB ports 354, the locking mechanism 384, and the display 360. The example display 360 is a touch screen display.

In some embodiments, the display 360 is configured to display a numerical value as a percentage of power remaining in the power supply. In the example power supply, the display 360 is illuminated by LED lights which can illuminate as green, yellow, or red based on the level of the power remaining. As will be described in greater detail later, a controller 420 within the power supply may monitor the power level of the power supply. The controller 420 can control the background illumination of the display 360 to provide an indication to a user of the current power remaining. For instance, the background can illuminate green to indicate a first power level threshold between a power level of 95-26%. A yellow illumination may indicate a second power level threshold of 25%-11%, while the red indicates a third power level threshold of 10% and below.

As described previously, external power source S can be one or more additional uninterruptible power supply systems 104 which are daisy chained to enlarge the available level of stored energy. When daisy chaining the uninterruptible power supply systems 104 together, the first uninterruptible power supply system 104 in the chain supplies power through bypasses of the uninterruptible power supply system 104 to the electronic medical device 102. The controller 420 of each power supply system 104 may determine if the power is not supplied from the power supply system 104 before each respective power supply system 104 in the chain. When a power supply system 104 detects a lack of received power, the power supply system 104 begins supplying power as the external power source S, while the remaining power supply systems 104 downstream continue to bypass power to the electronic medical device 102.

The example locking mechanism 384 is adjacent the AC output ports 352a, 352b on the back side 374. While shown on only the back side 374, it is understood the locking mechanism 384 may be placed adjacent any of the input port 350, output ports 352a, 352b, or USB ports 354 on the housing 320. The locking mechanism 384 may include a locking member 386 and a locking structure 388. As shown in FIG. 6, the example locking mechanism 384 is configured to allow a locking member 386 to hold a power cable 124 of the medical device within either one of AC output ports 134a, b and prevent removal while the locking member 386 is engaged. Referring to FIG. 8, in the example locking mechanism 384, the locking structure 388 is a locking loop integrally formed as part of the housing. In the example locking mechanism, the locking structure 388 is positioned near the input port 350 and output ports 352a, 352b adjacent to the top and bottom sides 378, 376 of the housing. In some examples, the locking structure 388 may be immediately adjacent the output ports 352a, 352b. In other examples the locking structure 388 may be offset from the output ports 352a, 352b at the bottom side 376. The locking member 386 may be a reusable zip tie; however, other ties or locking configurations may be used. The locking member 386, such as the zip tie, may be inserted through the locking structure 388 (i.e., the locking loop) and around a cable to hold the cable within a respective port. The zip tie may also be provided a color different from the cable and/or housing to allow the locking mechanism to be clearly visible.

In the example locking mechanism of FIG. 6, the locking structure 388 of the locking mechanism 384 may be a passageway 389 formed entirely within a shell of the housing 130. The passageway 389 forms a loop configured to allow the locking member 386 to be looped through the passageway and around a cable plugged into one of the input port 350 and output ports 352a, 352b. For instance, the passageway may be U-shaped with both ends accessible at the exterior of the housing 130. The passageway 389 does not enter the interior of the housing. In other examples, the locking mechanism 384 may be formed a plurality of locking loops 602 (shown best in FIGS. 16 and 17). Individual locking loops can be positioned at the front side 372 of the housing at positions both above and below the output ports 352a, 352b. A first locking loop can extend outward from the housing on a first side of the output port and a second locking loop can extend outward from the housing on a second side of the output port. Locking loops 602 can also be above and below the input ports 350 at the back side 374 of the housing. The locking loops 602 extend outward from the front side 372 in a direction parallel with a length of the housing. The locking member 386 (such as a zip tie) is inserted through the locking loop 602, at either one of or both of the locking loops 602 positioned above and below the ports 352a, 352b, and around the cable. Once wrapped around the cable, the locking member secures the cable within the port by locking against the loop 602 and preventing withdrawal of the cable. By having locking loops 602 on both sides of the input port 350 and output ports 352a, 352b, the cable can be secured to either one of locking loops 602 or can be secured to multiple loops on opposite sides of the port. Additionally, the two locking loop 602 configuration provides greater accessibility by providing a second side with a locking loop 602 in scenarios where one side of the housing is blocked and securing a locking member 386 may be more difficult to wrap a zip tie through than the other of the two locking loops 602. As shown in FIG. 17, the locking members 386 provide a force in the direction of the output port which prevents accidental removal of plugs from the output ports 352a, 352b.

FIG. 9 illustrates a handle on the uninterruptible power supply system 104. The handle allows a user to easily pick up the uninterruptible power supply system 104 while transporting a patient. Alternatively, the uninterruptible power supply system 104 can also be used to move the uninterruptible power supply system 104 when not in use.

FIG. 10 illustrates a bracket of the uninterruptible power supply system 104. The bracket may be used for mounting the uninterruptible power supply system 104 to either the medical device 102, a cart or other support structure. This allows the uninterruptible power supply system 104 to be easily held either during transport with the medical device or while in use.

FIG. 11 illustrates a cross sectional view showing the cooling system 390. The power supply system 104 also includes the cooling system 390 within an interior of the housing 130. The cooling system configured to use a solid state Peltier cooling system. The solid state Peltier cooling system is configured as a Peltier cooler 392. The Peltier cooler 392 is positioned between a bottom side 376 of the housing and the battery pack 330. Beneficially, heat is exchanged from the battery pack 330 to the housing 320 and is dissipated through the cooler 392. As such, no air openings or vents are in communication with the interior of the housing, which prevents the need to disinfecting the interior of the power supply 104 between uses of different patients in hospitals. In other examples, a fan 394 could be included to ventilate the interior of the housing 320 in addition to the Peltier cooler 392. In other examples, holes 396 (indicated in dotted lines to indicate possible locations) for ventilating the interior of the housing 320 may be provided instead of a fan. The fan 394 and holes 396 provide alternate methods of venting heat from the interior when sterilizing the power supply system 104 is not required. Fins 397 may be provided within the interior to help transfer heat from the battery pack 330 to the fan 394 or holes 396.

FIG. 12 illustrates an example block diagram of the uninterruptible power supply system 104 of the uninterruptible medical system 100. The example uninterruptible power supply system 104 includes a power supply 410, a controller 420, a power supply circuit 430, and a warning system 440.

The controller 420 is configured to control the supply of power to medical devices and activation of the warning system 440. The controller 420 is further configured to control switching the power supply between a first operation mode, a second operation mode, and a third operational. For instance, the controller 420 controls the power supply circuit and is configured to select between a pass-through mode, a back-up power mode, and an inverter mode. Accordingly, the controller 420 is in communication with the warning system 440, the display module 450 and the display 460.

The power supply circuit 430 can include a battery charger 432, a battery pack 434. The power supply circuit 430 allows the uninterruptible power supply 410 to bypass power directly to the electronic medical device 102 or allows the charging of the battery pack 434. The power supply circuit electrically coupled to the input port, output port, and battery pack 434. The battery charger 432 converts energy received from an external power source S to charge the battery pack 434. The input port 132 charges the uninterruptible power supply system 104 at 125-watts for 900-watt hours. The battery pack 434 provides a source of power for the electronic medical device 102 incase power to the external power source S is interrupted or lost. It is to be understood that the battery pack 434 can be the battery pack 330.

The battery charger 432 is connected to the battery pack 434 to receive and convert energy from the power supply circuit 430 to the battery pack 434. The warning system 440 is in communication with the controller 420, a display module 450, and an alarm module 470. The example display module 450 controls the operation of the display 460. The alarm module 470 is configured to control a plurality of alarms.

The power supply circuit 430 is configured to supply power based on a selected operational mode. The selected mode can include at least a first operational mode, a second operational mode, and third operational mode. The first operation mode is a pass-through mode, the second operation mode is a back-up power mode, and the third mode is an inverter mode. The pass-through mode allows the power from the power source S to pass-through the power supply 410 to the electronic medical device 102 and directly the power the medical device. The back-up power mode switches the power to be supplied from the power supply 410 to electronic medical device 102. The back-up mode can be either selected or the controller 420 can automatically switch to the back-up mode based on a lack of power received from the external power source. The inverter mode disconnects the pass-through path of the power supply circuit 430 and allows the battery pack 434 to be simultaneously charged by the external power source and discharged to the medical device. Additionally, the inverter mode may convert a square or simulated sine wave to a clean pure sine wave signal. As will be explained in great detailed below, the selection of the mode may be determined based on the interactions with the display. The third operational mode provides a mode to better meet transportation needs for disconnecting and reconnecting the power supply 104, while allowing the uninterruptible power supply to still operate in the first and second operation modes. The first and second operation modes provide less power loss caused by heat caused by heat generated when conversion of the between DC to AC during the inverter mode. A path for power supplied through the battery pack 434 and the inverter 480 may referred to as a battery pack path.

The display module 450 is configured to receive and send signals to operate different LED lights of the display 460 to be illuminated. The display module 450 is in communication with the controller 420. The illumination of LED lights can provide indications of power level of the battery and the detection of selections of the touch screen of the display. As described previously some examples may include LED lights which can be illuminated in different colors. In some instances, the different colors may indicate different threshold of power remaining within the battery pack 434. The display module 450 can also allow access to an engineering screen mode. The engineering screen mode is configured to allow access to operational data of the power supply 410. In particular, a manufacturer, a technician, or a user may perform a procedure to access the operational data. For instance, in the example display 360, a manufacturer may touch and hold a button 462 displayed on the touch screen for a predetermine amount of time. Once the predetermined amount of time has been reached, the engineering screen module signals the display module to display operational parameters. Non-limiting examples may include watt hours run, etc. In some instances, the button 462 may not actually be displayed or be visible; however, a user may still be able to press the area on the display associated with the button 462 to engage the engineering screen. Beneficially, users which know of the button 462 may engage the engineering screen and reduce the chance of other users starting the engineering screen. The display 460 may be communicate with the controller 420 and be used to switch between different operating modes. Non-limiting examples of the display switching between operating modes can include a drop down menu, a press and hold button, a plurality of buttons each corresponding for one of the three modes, and/or other known conventional methods of switching modes.

In the example power supply 410, the display 460 is illuminated by LED lights which can illuminate as green, yellow, or red based on the level of the power remaining. The controller 420 within the power supply 410 may monitor the power level of the power supply. The controller 420 may control the illumination of the LED lights to provide an indication to a user of the current power remaining. The controller 420 may send a signal to the warning system to switch the color of the LED lights. As a non-limiting example, the lights may illuminate green, yellow, or red. For instance, green light may indicate a first power level threshold between a power level of 95-26%. A yellow light may indicate a second power level threshold of 25%-11%, while the red indicates a third power level threshold of 10% and below. Further, the display 460 can include a speaker 464 which outputs alarms. In other examples, the display 360 may have a different interface which provides an indication of power draw from the power supply and has an alarm on/off function for all power level thresholds.

The alarm module 470 controls the plurality of alarms to warn users of low power levels of the uninterruptible power supply system 104 which if no charged may lead to operation of the electronic medical device 102 being interrupted. It should be understood that speakers may provide the alarms. Each alarm provides a different audible noise and indicating a different level of level of stored power. The audible noise is a continuous noise and may be mutable. In some instances, the audible noise may a different tone or quicker repetition of the audible noise. The warning system is configured to begin one of the alarms when a predetermined level of stored power is reached.

In some examples, a first alarm indicates a first predetermined level of power remaining, while a second alarm indicates a second predetermined level of power remaining lower than the first predetermined level. In some examples, a second button 466 may be pressed by a user to silence the first or second alarm. Preferably, both the first and second alarms are continuous until silenced by a user. In some examples, the second alarm may be quicker repeating tone and/or louder than the first alarm. In other instances, the second alarm is a different tone.

The warning system 440 further includes a lower limit alarm. When a lower limit of power remaining is reached, the lower limit alarm is triggered, and the lower limit alarm continues until the uninterruptible medical system 100 receives power from the external power source and the controller 420 prevent muting of the lower limit alarm by pressing the second button 466 (shown in FIG. 14). The lower limit alarm may also have a different tone, faster tone, and/or louder tone than the first and/or second alarm. In some examples, the alarm may be a repeatable recording. For instance, the recording may state to reconnect the battery pack 434 or that the battery pack 434 is critically low. The lower limit of power remaining may be 5% or below.

Because the lower limit alarm is unable to muted unless connection to an external power source is restored, the lower limit alarm reduces the likelihood the battery pack 434 runs out of power before being plugged into an external power source S. The lower limit alarm is particularly advantageous in medical transport scenarios where a patient may often be relying on life-sustaining medical devices which require power to be maintained without interruption. Further the uninterruptible power supply system 104 may switch to supply power within 12 ms of loss of power to an external power source. The example lower limit alarm provides a final alarm which cannot be silenced as a final warning, while providing multiple prior warnings of battery level. In some embodiments, the switching time is in a range from 6-24 ms. In another embodiment, the switching time is in a range from 12-16 ms. In yet another embodiment, the switching time is in a range from 5-9 ms. The switching time may be adjusted by changing a switch or relay used to change between the sources of power supply. For instance, a reduced distance between contacts of the switches, a quicker dissipation of the magnetic field, and mass of a switch armature can lead to quicker switch times. In other examples, the switch may have a flyback diode and quicker switch times can be achieved by connecting a zener diode in series with the flyback diode. Zener diodes conduct when a voltage is above a certain threshold which means during the peak of the reverse voltage spike when energy is the highest current is safely dissipated through the relay. However, once voltage reaches a safe threshold, conduction through the coil stops and the voltage across the relay coil can quickly drop to a release threshold. Examples of acceptable relays usable with the power supply are TE Relay Product Mini Relay K and Ong Chuan's Relay 792.

In some instances, the alarm module 470 may instead be configured to a be silent alarm. For example, instead of sounding an audible alarm, different colored lights, different illumination sequences, or other visual indicator may be used to alert a user of the amount of stored power remaining. In some examples, the silent alarm may be a default alarm setting. In other examples, the audible alarms may be the default alarm setting.

FIG. 13 illustrates an example block diagram of the power supply circuit 430 with certain elements of FIG. 12 not shown. The power supply circuit 430 includes a battery pack 434, the battery charger 432, a DC to AC inverter 480, an external power bypass 482, a voltage regulator 484, a surge suppressor 486, a switch 488, a switch 489, and a switch 490. The controller 420 is configured to open and close the switches 488, 489, 490. In some instances, the external power bypass 482 may be referred to as a pass-through path. In some examples, the term switch may alternatively be referred to as a relay.

When operating the first operational mode, power is passed through a closed switch 490 leading the external power bypass 482. The external power bypass 482 allows external power supplied at the input port 132 to bypass the battery and be used to directly power the electronic medical device 102. In the first operational mode, external power may be supplied through the external power bypass 482 to the output ports of the power supply 410. The voltage regulator 484 regulates the voltage at a fixed voltage. The surge suppressor 486 suppresses surges or spikes of voltage in the external power bypass 482. The controller 420 is in communication with the power supply circuit 430, input port 132, battery charger 432, battery pack 434 and a switch 488. The controller 420 is able to switch been supplying power received at the input port 132 to the external power bypass 482 or to the battery charger 432. In other instances of the first operational mode, the power supply circuit may be switched to supply power to the battery charger 432 to recharges the battery pack 434 and supply power to the electronic medical device 102. Additionally, the controller 420 is in communication with the switch 488 to switch between whether the power is supplied from the bypass 482 or battery pack 434 to the at least one output port 134. As such, the uninterruptible power supply system 104 of the uninterruptible medical system 100 operates as a line interactive uninterruptible power supply. The switch 489 remains closed and the switch 488 is not connected with to the battery pack path, allowing the battery pack is able to be recharged during the first operational mode but not provide power. In the second operational mode, the switch 488 connects the output port with the battery pack 434 to supply power from the battery pack 434 and a switch 490 is opened disconnecting the pass-through path. As such, the power supply 310 provides back-up power and utilizes battery power to power the electronic medical device 102 when either no external power source is connected, or a power outage is detected. In the third operational mode (i.e., the inverter mode), the controller 420 may operate a switch 488 and a switch 490 to disconnect the pass-through 482 and supply power from the battery pack 434. The switch 488 is joined with the battery pack path and the switch 490 is opened disconnecting the pass-through path entirely. As such a switch 489 connects power from the external power sources through the switch 488. In the third operational mode, the battery pack 434 may also receive power from the external power source S while discharging to the medical device. Generally, the switch 489 remains closed in all operational modes unless high voltage or low voltage thresholds are reached.

FIG. 14 is a front perspective view of the display of the uninterruptible power supply system 104. The display 460 sends signals to the controller 420 to activate the switching the between the first, second and third operational modes. The display 460 may include a third button 468 (shown in FIG. 14) displayed on the touch screen of the display 460 which allows the switching between the first operation mode, the second operation mode, and third operational mode. In some examples, the button 468 may be described as a mode switching button.

FIG. 15 is a flow diagram of a method of controlling the illumination of the display. The method 500 includes operations 505, 510, 515, 520, 525, 530, 535, 540, 545, 550, and 555. The operation 505 is performed to detect the power level of the battery. For instance, the controller may detect the power level by coulomb counting.

The operation 510 detects if the battery level is between a first range. An example range may be 95% to 25%. It is understood the first range may vary in other examples. If no, the method proceeds to operation 525. If yes, operation 515 illuminates the first light. The method proceeds to operation 520 which detects if the battery pack has been connected to an external power source. If the battery pack has been connected to an external power source, the method proceeds to operation 555 which determines if the battery pack has been recharged and returns to operation 505.

If the power is not within the first range as detected in operation 510, the method continues to operation 525. The operation 525 detects if the battery level is lower than a second range. An example range may be a range below 25% and higher than 10%. It is understood the second range may vary in other examples. If no, the method proceeds to operation 540. If yes, operation 530 illuminates the second light. The method then proceeds to operation 535 detects if the battery back has been connected to an external power source. In operation 535, it is detected if the battery back has been connected to an external power source, the method proceeds to operation 555 which determines if the battery pack has been recharged and returns to operation 505. If no, the method proceeds to operation 535.

If the power is not within the first range as detected in operation 525, the method continues to the operation 540. The operation 540 detects if the battery level is lower than a third range. An example range may be 10% or below. It is understood the third range may vary in other examples. If no, the method proceeds to operation 550. If yes, operations 545 illuminates the third light and proceeds to operation 550. In operation 550, it is detected if the battery back has been connected to an external power source, the method proceeds to operation 555 which determines if the battery pack has been recharged and returns to operation 505.

The present disclosure also relates to a method of transporting an uninterruptible power supply system. The method includes an operation of in a first location, selecting an inverter mode of an uninterruptible power supply. The method further includes an operation of disconnecting the uninterruptible power supply from an external power source. The method further includes an operation of transporting the uninterruptible power supply to a second location. The method further includes an operation of connecting the uninterruptible power supply to a second external power source or power receptacle.

The present disclosure also relates to a method of operating an uninterruptible power supply system. The method includes an operation of selecting an inverter mode of an uninterruptible power supply. The method further includes an operation of disconnecting a first switch delivering power from an external power source to a pass-through path and a second switch receiving power from the pass-through path. The second switch providing power to an electronic device. The method includes an operation of connecting a third switch delivering power from the external power source to a battery pack path and connecting the second switch to the battery pack path to provide power the electronic device.

The various examples described above are provided by way of illustration only and should not be construed to limit the scope of the present disclosure. Those skilled in the art will readily recognize various modifications and changes that may be made without following the example examples and applications illustrated and described herein, and without departing from the true spirit and scope of the present disclosure.

What is claimed is:

1. An uninterruptible power supply comprising:
a housing;
at least one input port configured to receive power from an external power source;
at least one output port configured to provide power to a medical device;
a battery pack contained within the housing;
a power supply circuit electrically coupled to the input port, the output port, and the battery pack; and
a controller that controls the power supply circuit and is configured to select between a pass-through mode, a back-up power mode, and an inverter mode.

2. The uninterruptible power supply of claim 1, wherein the pass-through mode allows the passthrough of power from the external power source and wherein the back-up power mode supplies power from the battery pack directly to the medical device.

3. The uninterruptible power supply of claim 1, wherein the inverter mode is configured to disconnect a pass-through path of the power supply and allows the battery pack to be simultaneously charged by the external power source and discharged to the medical device.

4. The uninterruptible power supply of claim 1, further including a touch screen display.

5. The uninterruptible power supply of claim 4, wherein the touch screen display is configured to display a mode switching button, wherein the mode switching button configured to signal the controller to switch between the pass-through mode, the back-up power mode, and the inverter mode.

6. The uninterruptible power supply of claim 5, wherein the touch screen display is configured to display an engineering screen, and wherein the engineering screen displays operating parameters of the power supply.

7. The uninterruptible power supply of claim 6, wherein the operating parameters include Watt hours.

8. The uninterruptible power supply of claim 7, wherein the engineering screen is activated when the controller detects the mode switching button has been pressed and held for longer than a threshold amount of time.

9. The uninterruptible power supply of claim 4, wherein the touch screen display is illuminated by LED lights, and wherein the touch screen display illuminates a plurality of based on a detected power remaining in the power supply, and wherein each color indicates a different range of power remaining within the power supply.

10. The uninterruptible power supply of claim 1, wherein the housing includes at least one locking mechanism configured to hold a cable within at least one of the input port and the output port.

11. The uninterruptible power supply of claim 10, wherein the at least one locking mechanism is a locking loop adjacent to at least one of the input port and output port and extending outward from a side of the housing.

12. The uninterruptible power supply of claim 11, wherein the locking loop is a first locking loop and a second locking loop adjacent to the output port, the first locking loop extending outward from the housing on a first side of the output port and the second locking loop extending outward from the housing on a second side of the output port.

13. The uninterruptible power supply of claim 1, wherein the housing further comprises a handle.

14. The uninterruptible power supply of claim 1, wherein the housing further comprises a fuse panel configured to movably allow or restrict access to a fuse of the power supply and wherein the fuse panel is configured to allow the replacement of the fuse without disassembling the housing.

15. The uninterruptible power supply of claim 1, wherein the housing including a first half and a second half, the first half and second half joined together and wherein the first half and second half are joined together by both tabs and a plurality of fasteners.

16. The uninterruptible power supply of claim 1, wherein the power supply weighs no more than 17 lbs and provides at least a peak of 1000 W.

17. The uninterruptible power supply of claim 1, wherein the power supply weighs no more than 17 lbs and provides at least a peak of 2400 W.

18. The uninterruptible power supply of claim 1, wherein the housing comprises a fire retardant material and the fire retardant material meets UL94 V0 ratings.

19. The uninterruptible power supply of claim 1, wherein the housing does not include openings to the interior.

20. The uninterruptible power supply of claim 1, wherein when the power falls below a threshold, a silent alarm is turned on.
